(19) United States
(12) Patent Application Publication
(10) Pub. No.: US 2010/0056583 A1
(43) Pub. Date: Mar. 4, 2010

(54) Polymorphic forms of rosiglitazone hydrobromide and processes for their preparation

(75) Inventors: Vinod Kumar Kansal, Haryana (IN); Harish Ranjan, New Delhi (IN); Sanjay Nayal, Haryana (IN); Michal Rafilovich, Petach-Tikva (IL)

Correspondence Address: KENYON & KENYON LLP, ONE BROADWAY, NEW YORK, NY, 10004, US

(73) Assignees: TEVA PHARMACEUTICAL INDUSTRIES LTD., Petah Tiqva (IL); TEVA PHARMACEUTICALS USA, INC. - for assignment of rights in Barbados, North Wales, PA (US)

(21) Appl. No.: 12/583,509

(22) Filed: Aug. 21, 2009

Related U.S. Application Data
(60) Provisional application No. 61/090,738, filed on Aug. 21, 2008.
(60) Provisional application No. 61/146,500, filed on Jan. 22, 2009.
(60) Provisional application No. 61/163,187, filed on Mar. 25, 2009.

(51) Int. Cl. A61K 31/4439; C07D 417/02; A61P 3/10

(52) U.S. Cl. 514/342; 546/269.7

(57) ABSTRACT

The present invention provides crystalline forms of Rosiglitazone hydrobromide, methods of their preparation, as well as pharmaceutical compositions comprising these crystalline forms.

### CROSS REFERENCE TO RELATED APPLICATIONS

The present invention claims the benefit of the following U.S. Provisional Patent Application Nos. 61/090,738, filed Aug. 21, 2008; 61/146,500, filed Jan. 22, 2009; and 61/163,187, filed Mar. 25, 2009. The contents of these applications are incorporated herein by reference.

### FIELD OF THE INVENTION

The present invention is related to the solid state chemistry of Rosiglitazone hydrobromide, specifically, crystalline forms of Rosiglitazone hydrobromide salt, as well as to methods for their preparation.

### BACKGROUND OF THE INVENTION

Rosiglitazone, (5-((4-(2-(methyl-2-pyridinylamino)ethoxy)phenyl)methyl)-2,4-thiazolidinedione, is an anti-diabetic drug from the thiazolidinedione class which acts primarily by increasing insulin sensitivity. This molecule has the following structure:

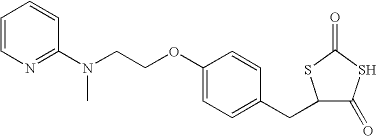

International Patent Application No. WO94/05659, describes salts of Rosiglitazone, including Rosiglitazone maleate. Rosiglitazone maleate is marketed in the U.S. under the trade name: AVANDIA® in 2 mg, 4 mg, and 8 mg tablets. Rosiglitazone and its meleate salt are described in the following U.S. patents, hereby incorporated by reference: U.S. Pat. Nos. 5,002,953; 5,741,803; and 6,288,095.

Rosiglitazone hydrobromide salt is described in International Patent Application No. WO 01/94344. This salt is characterized by IR, Raman, X-Ray powder diffraction, solid state ¹³C-NMR and a melting point. Crystalline forms of this salt are apparently produced by reacting Rosiglitazone or a salt thereof, dissolved in a suitable solvent, with a source of hydrogen bromide, following by its recovery. In the examples of WO 01/94344, propan-2-ol and acetone are used.

U.S. Publication No. 2008/0176905 describes crystalline forms I and II of Rosiglitazone hydrobromide.

In addition, U.S. Publication No. 2006/0083784 described amorphous Rosiglitazone hydrobromide.

The present invention relates to the solid state physical properties of Rosiglitazone hydrobromide. These properties can be influenced by controlling the conditions under which Rosiglitazone hydrobromide is obtained in solid form. Solid state physical properties include, for example, the flow-ability of the milled solid. Flow-ability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper,; limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetric (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

One of the most important physical properties of a pharmaceutical compound, which can form polymorphs or solvates, is its solubility in aqueous solution, particularly the solubility in gastric juices of a patient. Other important properties relate to the ease of processing the form into pharmaceutical dosages, as the tendency of a powdered or granulated form to flow and the surface properties that determine whether crystals of the form will adhere to each other when compacted into a tablet.

The discovery of new polymorphic forms and solvates of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

Thus, there is a need in the art for new polymorphs of Rosiglitazone hydrobromide and processes for the preparation of Rosiglitazone hydrobromide forms.

### SUMMARY OF THE INVENTION

In one embodiment, the present invention provides crystalline Rosiglitazone hydrobromide Form I, characterized by a powder XRD pattern having peaks at about 15.2, 17.8, 22.9 and 24.5±0.2 degrees two-theta, having a water content of between 4.2-5.8% by weight.

The present invention encompasses a crystalline form of Rosiglitazone HBr, designated Form III, characterized by data selected from the group consisting of: a powder XRD pattern having peaks at about 4.5, 10.9, 17.0, 18.3, and 19.1±0.2 degrees two-theta; a powder XRD pattern with three peaks at about 10.9, 18.3 and 19.1±0.2 degrees two-theta and two peaks selected from the list of five peaks at about 4.5, 14.4, 16.3, 17.0 and 17.9±0.2 degrees two-theta; a powder XRD pattern with peaks at about 4.5, 10.9 and 19.1±0.2 degrees two-theta and additional peaks at about 16.3, 14.4, 17.0 and 17.9±0.2 degrees two-theta; a powder XRD pattern as depicted in FIG. 1; and combinations thereof. Preferably, the crystalline form of Rosiglitazone HBr form III has a crystalline purity of at least 80% by weight.

The present invention encompasses a crystalline form of Rosiglitazone HBr, designated Form IV, characterized by data selected from the group consisting of a powder XRD pattern having peaks at about 5.4, 9.1, 10.7, 15.0, and 18.3±0.2 degrees two-theta; a powder XRD pattern with five peaks at about 5.4, 9.1, 10.7, 15.0 and 18.3±0.2 degrees two-theta and additional four peaks at about 7.8, 14.8, 20.0 and 29.0±0.2 degrees two-theta; a powder XRD pattern as depicted in FIG. 2; and combinations thereof. Preferably, the crystalline form of Rosiglitazone HBr form IV has a crystalline purity of at least 80% by weight.

The present invention further encompasses processes for preparing the Rosiglitazone hydrobromide Forms III and IV.

The present invention also encompasses a process for preparing Rosiglitazone hydrobromide Form I comprising heating a solution of Rosiglitazone base and HBr in acetone; cooling the solution until a precipitate is obtained; and isolating the precipitate to obtain Rosiglitazone HBr Form I.

### BRIEF DESCRIPTION OF THE FIGURES

FIG. 1 illustrates a powder X-ray diffraction pattern for Rosiglitazone hydrobromide Form III.

FIG. 2 illustrates a powder X-ray diffraction pattern for Rosiglitazone hydrobromide Form IV.

FIG. 3 illustrates a powder X-ray diffraction pattern for Rosiglitazone hydrobromide Form I.

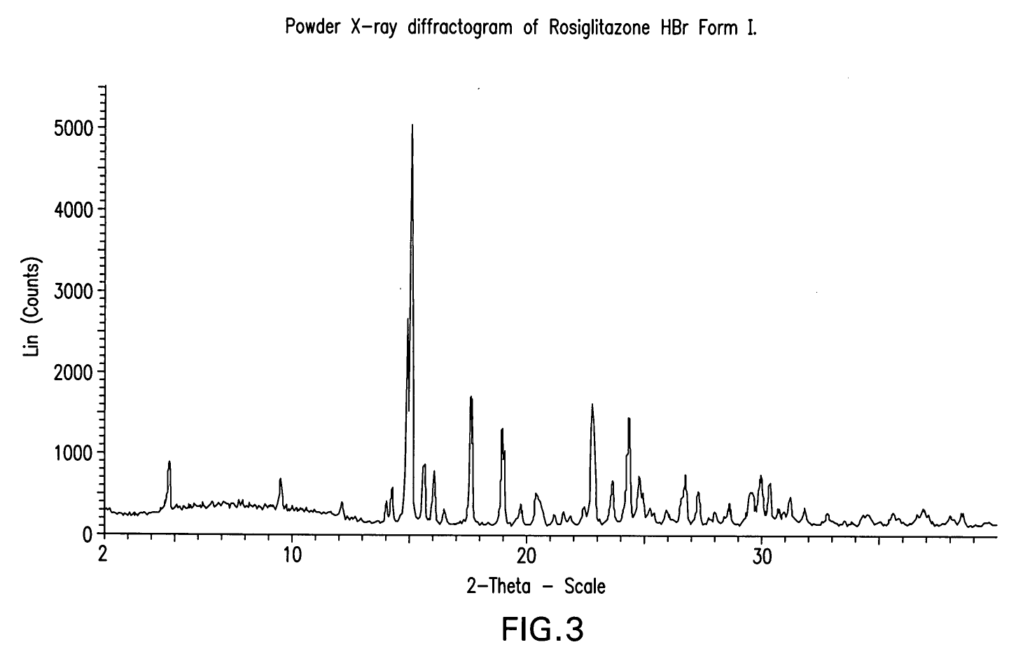

### DETAILED DESCRIPTION

The present invention provides novel crystalline forms of Rosiglitazone hydrobromide.

As used herein in connection with a measured quantity, the term “about” refers to that variation in the measured quantity as would be expected by the skilled artisan performing the measurement and exercising a level of care commensurate with the objective of the measurement and the precision of the measuring apparatus being used.

As used herein, the term “room temperature” refers to a temperature of about 20° C. to about 30° C., preferably about 25° C.

As used herein, the term volume (“V”) refers to ml per gram. For example, 30 V of solvent means 30 ml solvent per one gram of solid/compound, here for example Rosiglitazone base.

As used herein, the term “Rosiglitazone base Form I” refers to a crystalline Rosiglitazone base characterized by a powder XRD pattern having peaks at about 13.9, 16.6, 17.3, 21.9, 22.4, 25.3, 27.5, and 30.0±0.2 degrees two-theta. The Rosiglitazone base Form I can be prepared according to any method known in the art, for example, according to the procedure described in WO2008/017398, wherein the process for the preparation of polymorphic form I of rosiglitazone base comprises (i) dissolving rosiglitazone base in an organic solvent at a temperature in the range of from 20° C. to reflux temperature of said organic solvent; (ii) optionally adding an anti-solvent; (iii) cooling to a temperature in the range of from −10° C. to 30° C. and (iv) separating and drying of Polymorphic form I of rosiglitazone base. The organic solvent used herein is selected from the group consisting of an alcohol, an ester, an ether, a ketone and tetrahydrofurane (THF), and the anti-solvent may be selected from the group consisting of water and an organic antisolvent, such as an ether or toluene.

As used herein, the term “Rosiglitazone HBr Form I” refers to a crystalline Rosiglitazone HBr Form I characterized by a powder XRD pattern having peaks at about 15.2, 17.8, 22.9 and 24.5±0.2 degrees two-theta. The Rosiglitazone HBr Form I can be prepared according to any method known in the art, for example, according to the procedure described in the U.S. Publication No. 2008/0176905, wherein the process for preparing the Rosiglitazone hydrobromide form I comprises; providing a mixture of Rosiglitazone and a solvent selected from the list consisting of methyl ethyl ketone, C₃₋₇ esters, water and mixture thereof; and admixing the mixture with a source of hydrobromide; and cooling, preferably to a temperature of about 0° C. to about 60° C. to obtain a precipitate.

As used herein, the term “Rosiglitazone hydrobromide” includes any solid state composition of Rosiglitazone base and hydrobromic acid. For example: a salt, a co-crystal, or a solid mixture of base and acid. Preferably, “Rosiglitazone hydrobromide” refers to a Rosiglitazone hydrobromide salt, in which Rosiglitazone and hydrobromic acid are present in a molar ratio of about 1:1.

As used herein, the term “water content” refers to the content of water based upon the Loss on Drying method (the “LOD” method) as described in UPS 29-NF 24, official Aug. 1, 2006, Physical Test and Determinations, <731> LOSS ON DRYING or in Pharmacopeial Forum, Vol. 24, No. 1, p. 5438 (January-February 1998), the Karl Fisher assay for determining water content or thermogravimetric analysis (TGA). All percentages herein are by weight unless otherwise indicated.

Any one skilled in the art would understand that the term “Rosiglitazone hydrobromide hydrate” refers to Rosiglitazone hydrobromide having a water content of about 0.5% to about 6.0 % w/w; and that the term “Rosiglitazone hydrobromide monohydrate” refers to Rosiglitazone hydrobromide having a water content of about 3.5% to about 4.5% w/w.

As used herein the term “crystalline purity,” refers to a particular crystalline form of a compound in a sample which may contain amorphous form of the compound, one or more other crystalline forms of the compound other than the crystalline form of the compound of this invention, or a mixture thereof wherein the particular crystalline form of the compound is present in an amount of at least about 80%, preferably at least about 95%, more preferably at least about 99% crystalline, and most preferably about 100%.

The present invention describes crystalline Rosiglitazone hydrobromide Form I, characterized by a powder XRD pattern having peaks at about 15.2, 17.8, 22.9 and 24.5±0.2 degrees two-theta, having a water content of between 4.2-5.8% by weight. Preferably, the crystalline form of Rosiglitazone HBr form I has a crystalline purity of at least 80% by weight, more preferably having a crystalline purity of at least about 90% by weight, even more preferably having a crystalline purity of at least about 95% by weight.

The present invention provides a crystalline form of Rosiglitazone HBr, designated Form III. Preferably, Rosiglitazone HBr Form III is a hydrate, more preferably, a monohydrate.

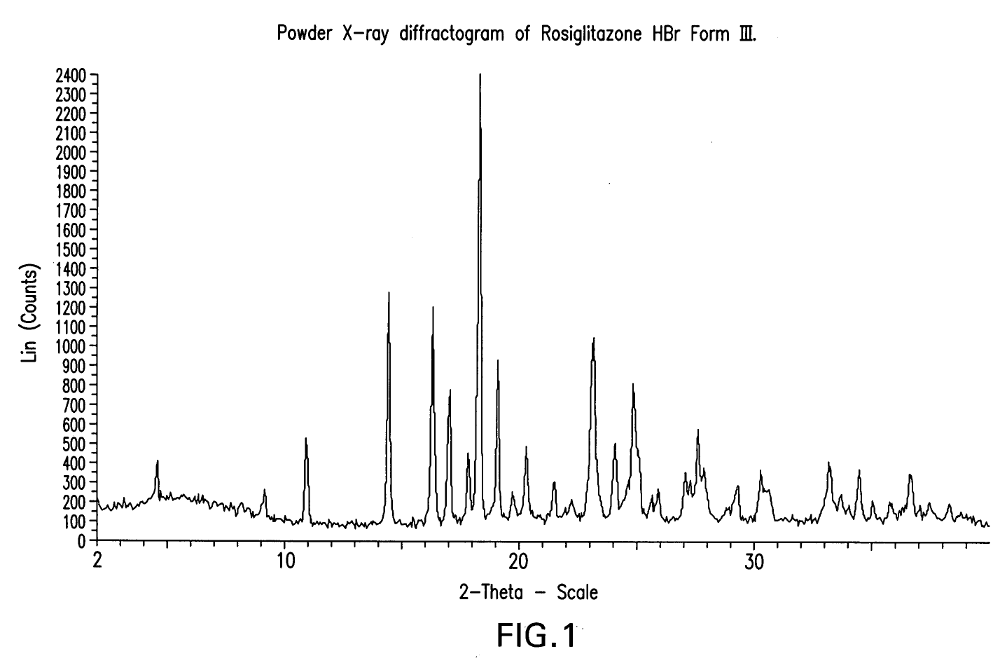

In one embodiment, the present invention encompasses Rosiglitazone HBr Form III, characterized by data selected from the group consisting of: a powder XRD pattern having peaks at about 4.5, 10.9, 17.0, 18.3, and 19.1±0.2 degrees two-theta; a powder XRD pattern with three peaks at about 10.9, 18.3 and 19.1±0.2 degrees two-theta and two peaks selected from the list of five peaks at about 4.5, 14.4, 16.3, 17.0 and 17.9±0.2 degrees two-theta; a powder XRD pattern with peaks at about 4.5, 10.9 and 19.1±0.2 degrees two-theta and additional peaks at about 16.3, 14.4, 17.0 and 17.9±0.2 degrees two-theta; a powder XRD pattern as depicted in FIG. 1; and combinations thereof.

Form III can be characterized by having a water content of 3.5% to about 4.5% water, by weight, which corresponds to Rosiglitazone HBr hydrate, preferably a monohydrate, which is expected to be more stable, compared to the anhydrous form, upon use in formulation processes. In addition, Form III has an irregular shape that may prevent various problems during manufacturing. For example, plate shaped crystals might have a propensity to aggregate one on top of the other and thus can cause solvent molecules to be trapped within the aggregates, and thus prevent removal of all the reaction solvent to be filtered out from the solid.

Preferably, the crystalline form of Rosiglitazone HBr form III has a crystalline purity of at least 80% by weight, more preferably having a crystalline purity of at least about 90% by weight, even more preferably having a crystalline purity of at least about 95% by weight.

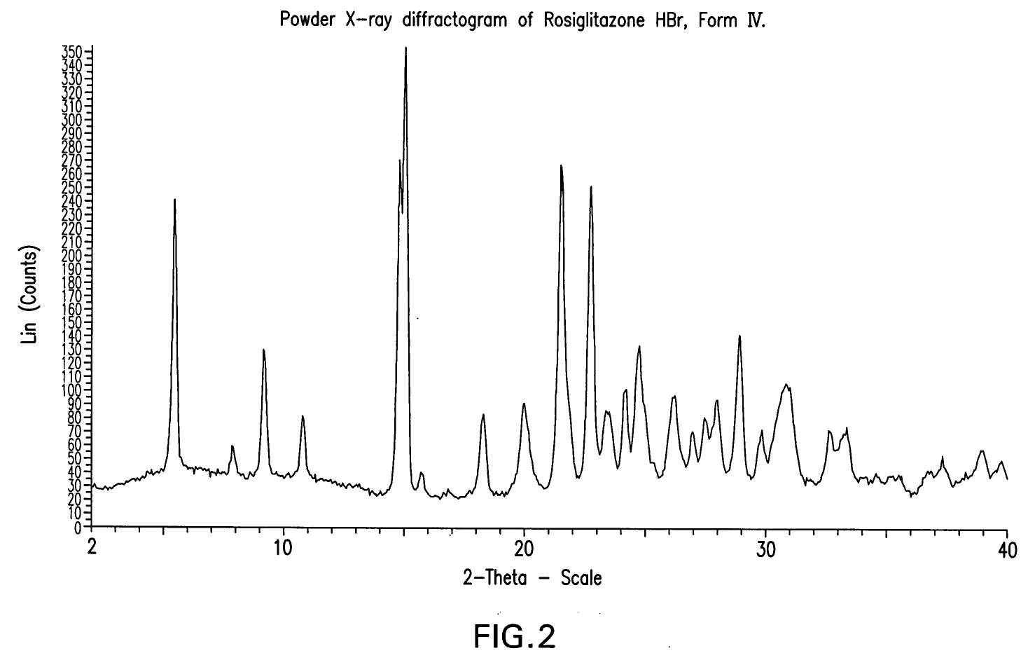

In another embodiment, the present invention encompasses a crystalline form of Rosiglitazone HBr, designated Form IV, characterized by data selected from the group consisting of a powder XRD pattern having peaks at about 5.4, 9.1, 10.7, 15.0, and 18.3±0.2 degrees two-theta; a powder XRD pattern with five peaks at about 5.4, 9.1, 10.7, 15.0 and 18.3±0.2 degrees two-theta and additional four peaks at about 7.8, 14.8, 20.0 and 29.0±0.2 degrees two-theta; a powder XRD pattern as depicted in FIG. 2; and combinations thereof.

Form IV is characterized by high degree of disordered lattice. This particular arrangement of molecules may give rise to good aqueous solubility, and therefore, may lead to a higher bioavailability.

Preferably, the crystalline form of Rosiglitazone HBr form IV has a crystalline purity of at least 80% by weight, more preferably having a crystalline purity of at least about 90% by weight, even more preferably having a crystalline purity of at least about 95% by weight.

The present invention encompasses a process for preparing Rosiglitazone hydrobromide Form I comprising heating a solution of Rosiglitazone base and HBr in acetone; cooling the solution until a precipitate is obtained; and isolating the precipitate to obtain Rosiglitazone HBr Form I. Preferably, about 8 volumes (V) to about 35 volumes (V) of solvent are applied, more preferably about 10V to about 20V, and most preferably, about 15V to about 18V.

Preferably, the solution is heated to a temperature of about 50° C. to about 65° C., more preferably about 55° C. to about 62° C., most preferably to about reflux temperature. Preferably, the solution is cooled to about 0° C. to about 45° C., more preferably, about room temperature.

Preferably, the HBr is added as an aqueous solution, for example a 48% w/w solution of hydrogen bromide in water, to a solution of Rosiglitazone base in acetone. Preferably, the HBr solution is added over a period of about 30 minutes to about 45 minutes. Alternatively, the source of hydrogen bromide is a solution of hydrogen bromide in acetone. Alternatively, the hydrogen bromide may be added directly to a solution of Rosiglitazone base in acetone. Another alternative source of hydrogen bromide may be hydrobromic acid (gas), or a base salt of hydrobromic acid for example ammonium bromide, or the hydrobromic acid salt of an amine (such as a C₁-C₉ amine), for example ethylamine or diethylamine. Preferably, the hydrogen bromide source is added drop-wise to the solution containing Rosiglitazone.

The obtained Rosiglitazone hydrobromide Form I may then be recovered. Recovery can be carried out by filtration. The recovered Rosiglitazone can be dried. Drying can be carried out under a pressure of less than one atmosphere (reduced pressure), including a pressure of less than about 100 mmHg. Drying can also be carried out by heating, with or without reducing the pressure. Heating can be carried out at room temperature to about 80° C., more preferably from room temperature to about 60° C., and even more preferably at about 40° C. to about 60° C.

The present invention further encompasses a process for preparing the Rosiglitazone hydrobromide Form III by a process comprising: providing a mixture of Rosiglitazone and water; heating the mixture to about 90° C. to about 105° C., more preferably, to about reflux; admixing the mixture with a source of hydrobromide to provide a solution; and cooling, preferably to a temperature of about 0° C. to about 60° C. or more preferably to about room temperature, to obtain a precipitate.

Preferably, 25-55 g of Rosiglitazone is used, and more preferably, 45-55 g of Rosiglitazone is used in the above described process. More preferably, about 50 g is used. Preferably, Rosiglitazone base is used at a concentration of about 50 mg/ml to about 110 mg/ml, more preferably about 90 mg/ml to about 110 mg/ml, most preferably at about 100 mg/ml.

The source of hydrogen bromide used may be an aqueous solution of hydrogen bromide, for example a 48% w/w solution of hydrogen bromide in water. Alternatively, the hydrogen bromide may be added directly to the mixture of Rosiglitazone in water. Another alternative source of hydrogen bromide may be hydrobromic acid (gas), or a base salt of hydrobromic acid for example ammonium bromide, or the hydrobromic acid salt of an amine (such as a C₁-C₉ amine), for example ethylamine or diethylamine. Preferably, the hydrogen bromide source is added drop-wise to the solution containing Rosiglitazone.

The obtained Rosiglitazone hydrobromide Form III may then be recovered. Recovery can be carried out by filtration. The recovered Rosiglitazone can be dried. Drying can be carried out under a pressure of less than one atmosphere (reduced pressure), including a pressure of less than about 100 mmHg. Drying can also be carried out by heating, with or without reducing the pressure. Heating can be carried out at room temperature to about 60° C., more preferably at about 40° C. to about 60° C.

The present invention further encompasses a process for preparing the Rosiglitazone hydrobromide Form IV by a process comprising: wetting Rosiglitazone hydrobromide Form I, preferably using between about 0.5 mL to about 1.5 mL of acetone per about 100 mg to about 200 mg Rosiglitazone HBr Form I, most preferably per about 150 mg of Rosiglitazone HBr form I, to obtain a paste of Rosiglitazone hydrobromide and acetone and further maintaining the obtained paste at a temperature of about 25° C. to about 45° C., preferably, for a period of about 18 hours to about 36 hours, more preferably for about 24 hours, to obtain Rosiglitazone hydrobromide Form IV, preferably a crystalline pure Rosiglitazone form IV. Preferably, the acetone contains less than 10%, preferably less than 5%, and most preferably, less than 2% water by weight.

Preferably, the temperature used when maintaining the paste is about 30° C. to about 40° C.

In yet another process, Rosiglitazone hydrobromide Form IV is prepared by a process comprising admixing Rosiglitazone hydrobromide Form I and acetone to obtain a suspension, and recovering the Rosiglitazone hydrobromide Form IV from the suspension, preferably a crystalline pure Rosiglitazone form IV. Preferably, the acetone contains less than 10%, preferably less than 5%, and most preferably, less than 2% water by weight.

Preferably, the Rosiglitazone hydrobromide Form I is admixed with the acetone at a temperature of about 25° C. to about 40° C., preferably about 30° C., while stirring. Preferably, the ratio of Rosiglitazone hydrobromide Form I to acetone is about 1:60 to about 1:100, preferably about 1:80 of Rosiglitazone hydrobromide Form I in grams to acetone in ml (w/v). Preferably the mixture is maintained at a temperature of about 25° C. to about 40° C. for a period of about 18 hours to about 36 hours, preferably for about 24 hours.

Recovery of Rosiglitazone hydrobromide Form IV can be performed according to conventional methods known in the field, such as vacuum filtering.

The present invention comprises 1) a pharmaceutical composition comprising any one, or combination of Rosiglitazone hydrobromide crystalline forms I, III or IV described above and at least one pharmaceutically acceptable excipient; and 2) the use of any one, or combination, of the above-described Rosiglitazone hydrobromide crystalline forms I, III or IV, in the manufacture of a pharmaceutical composition, wherein the pharmaceutical composition can be useful for the treatment of diabetes mellitus.

The pharmaceutical composition of the present invention can be in solid or a non-solid form. If the pharmaceutical composition is in a non-solid form, any one, or combination, of the Rosiglitazone hydrobromide crystalline forms I, III or IV are retained as solid(s) in the non-solid pharmaceutical composition e.g., as a suspension, foam, ointment and etc.

The pharmaceutical composition can be prepared by a process comprising combining any one, or combination of the above-described Rosiglitazone hydrobromide crystalline forms I, III or IV with at least one pharmaceutically acceptable excipient. The Rosiglitazone hydrobromide crystalline forms I, III or IV can be obtained by any of the processes of the present invention as described above.

The pharmaceutical composition can be used to make appropriate dosage forms such as tablets, powders, capsules, suppositories, sachets, troches, and lozenges.

The present invention provides a composition comprising at least 80%, preferably at least 90%, more preferably at least 95% and most preferably at least 99% by weight of one crystalline form of Rosiglitazone hydrobromide selected from Rosiglitazone hydrobromide Form III, Form IV, and Form I. The composition preferably contains 20% or less, preferably 10% or less, more preferably 5% or less, and most preferably 1% or less by weight of any other crystalline or amorphous form of Rosiglitazone hydrobromide.

Any one, or combination, of the above-mentioned Rosiglitazone hydrobromide crystalline forms I, III or IV, particularly in a pharmaceutical composition and dosage form, can be used to treat diabetes mellitus in a mammal such as a human, comprising administering a treatment effective amount of the one, or combination, of the Rosiglitazone hydrobromide crystalline forms I, III or IV in the mammal. The treatment effective amount or proper dosage to be used can be determined by one of ordinary skill in the art, which can depend on the method of administration, the bioavailability, the age, sex, symptoms, and health condition of the patient, and the severity of the disease to be treated, etc.

Having described the invention with reference to certain preferred embodiments, other embodiments will become apparent to one skilled in the art from consideration of the specification. The disclosures of the references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### Examples

### Instruments

### XRD

Powder X-ray diffraction (“XRD”) analysis can be carried out using any XRD powder diffractometer commonly used in the industry. The Rosiglitazone hydrobromide samples of this invention were run in a Bruker D8 Advance, X-Ray powder diffractometer, Cu-tube with radiation wavelength of λ=1.5418 Å, LynxEye position sensitive detector. A standard sample holder of PMMA (SN: C79298A3244D82/84) or PMMA with silicon low background plate, Ø51.5 mm, with Ø20 mm*0.5 mm sample cavity (SN:C79298A3244B261).

### Water Content

Water content was determined by Karl Fisher analysis using Mettler Toledo DL 38 Karl Fisher Titrator.

### Example 1

### Process for the Preparation of Rosiglitazone HBr, Form III

A 1000 ml flask was charged with Rosiglitazone (50 g) and demineralized water (500 ml). The suspension was heated to reflux and then aqueous Hydrobromic acid 47-48% was added (20 ml) drop wise to obtain a clear solution, stirred for an additional 30-45 minutes and cooled to room temperature while stirring for 19-20 hours. The resulting solid was filtered under reduced pressure and washed by DM (demineralized) water (50 ml). Finally dried at 48-50° C. under reduced pressure to give 52 g (85%) of a white solid. The resulting solid was analyzed by XRD to yield Rosiglitazone hydrobromic acid Form III.

### Example 2

### Formulation of Rosiglitazone HBr, Form III

Solid pharmaceutical compositions of Rosiglitazone HBr Form III and the following excipients were compacted into a dosage form like a tablet: lactose monohydrate, sucrose and avicel.

### Example 3

### Process for the Preparation of Rosiglitazone HBr, Form IV

150 mg of Rosiglitazone HBr Form I was placed into plastic cup of 100 ml volume and wetted by 1 ml of analytical acetone. The cup was then closed by its cover and placed in water bath that was heated to 30° C. for 24 hrs. The resulting solid was analyzed by PXRD and showed Rosiglitazone HBr Form IV.

### Example 4

### Process for the Preparation of Rosiglitazone HBr, Form IV

150 mg of Rosiglitazone HBr Form I was placed into plastic cup of 100 ml volume and wetted by 1 ml of analytical acetone. The cup was then closed by its cover and placed in water bath that was heated to 40° C. for 24 hours. The resulting solid was analyzed by PXRD and showed Rosiglitazone HBr Form IV.

### Example 5

### Process for the preparation of Rosiglitazone HBr, Form IV

150 mg of Rosiglitazone HBr Form I was placed into glass vial of 25 ml volume. Afterwards 12 ml analytical acetone was added to the vial with magnetic stirrer. The glass was then closed by its cover. The slurry was stirred for 24 hours in a water bath that was heated to 30° C. After 24 hours. The suspension was vacuum filtered for 0.5 hours. The resulting solid was analyzed by PXRD and showed Rosiglitazone HBr Form IV.

### Example 6

### Process for the Preparation of Rosiglitazone HBr, Form I

16.0 L Acetone was added to Rosiglitazone Free Base Form I (1.0 kg) at room temperature and heated to reflux temperature. The solution was stirred for 30 minutes at reflux temperature and 0.40 Liter HBr (47-48% aqueous solution) were added over a period of 30 to 45 minutes. The mixture was stirred for 30 minutes at reflux temperature. The reaction mixture was cooled to 45° C. over a period of 1.5 hours and stirred 1.5 hours at 45 to 40° C. The contents cooled from 40 to 20° C. over a period of 1.5 hours and were stirred for 3 hours at 18 to 22° C. The solid was filtered and washed twice with 1.0 Liter chilled acetone (5 to 10° C.). The product was dried under vacuum (15-25 mm/Hg) at 50° C. Yield: 1.10 kg

### Example 7

### Water Content of Rosiglitazone HBr Form I

Form I was stored between 0-100% relative humidity at room temperature for 7 days as indicated in Table 1, and confirmed to remain Form I by XRPD.

TABLE 1

| | KF (Karl | |
|---|---|---|
| % RH | Fisher) | |
| (Relative Humidity) | [%] | Form |
| Room conditions | 4.2 | I |
| 0 | 0.7 | I |
| 40 | 4.2 | I |
| 60 | 4.2 | I |
| 80 | 4.2 | I |
| 100 | 5.8 | I |

1. Rosiglitazone HBr Form IV, characterized by data selected from the group consisting of a powder XRD pattern having peaks at about 5.4, 9.1, 10.7, 15.0, and 18.3±0.2 degrees two-theta; a powder XRD pattern with five peaks at about 5.4, 9.1, 10.7, 15.0 and 18.3±0.2 degrees two-theta and additional four peaks at about 7.8, 14.8, 20.0 and 29.0±0.2 degrees two-theta; a powder XRD pattern as depicted in FIG. 2; and combinations thereof.

2. The Rosiglitazone HBr of claim 1, characterized by a powder XRD pattern having peaks at about 5.4, 9.1, 10.7, 15.0, and 18.3±0.2 degrees two-theta.

3. The Rosiglitazone HBr of claim 1, having an X-ray diffraction diagram substantially as depicted in FIG. 2.

4. The Rosiglitazone HBr of claim 1, having a crystalline purity of at least about 80% by weight.

5. The Rosigilitazone HBr of claim 4, wherein the crystalline purity is at least about 90% by weight.

6. A process for preparing Rosiglitazone HBr Form IV of claim 1 comprising wetting Rosiglitazone hydrobromide Form I using acetone to obtain a paste of hydrobromide and acetone and further maintaining the obtained paste at a temperature of about 25° C. to about 45° C. to obtain Rosiglitazone hydrobromide Form IV.

7. The process of claim 6, wherein the obtained Rosiglitazone HBr Form IV has a crystalline purity of at least about 80% by weight.

8. The process of claim 6, wherein the temperature used when maintaining the paste is about 30° C. to about 40° C.

9. The process of claim 6, wherein the paste is maintained for about 18 hours to about 36 hours.

10. The process of claim 6, wherein wetting Rosiglitazone hydrobromide Form I is with about 0.5 mL to about 1.5 mL of acetone per about 100 mg to about 200 mg of Rosiglitazone HBr.

11. A process for preparing Rosiglitazone HBr Form IV comprising admixing Rosiglitazone hydrobromide Form I and acetone to obtain a suspension, and recovering the Rosiglitazone hydrobromide Form IV from the suspension.

12. The process of claim 11, wherein the obtained Rosiglitazone HBr Form IV has a crystalline purity of at least about 80% by weight.

13. The process of claim 11, wherein Rosiglitazone hydrobromide Form I is admixed with acetone at a temperature of about 25° C. to about 40° C.

14. The process of claim 11, wherein the ratio of Rosiglitazone hydrobromide Form I to acetone is about 1:60 to about 1:100 of Rosiglitazone hydrobromide Form I in grams to acetone in ml (w/v).

15. A process for preparing Rosiglitazone HBr Form I comprising heating a solution of Rosiglitazone base and HBr in acetone; cooling the solution until a precipitate is obtained; and isolating the precipitate to obtain Rosiglitazone HBr Form I.

16. The process of claim 15, wherein the amount of acetone is about 8 volumes (V) to about 35 volumes (V).

17. The process of claim 15, wherein the solution is heated to about reflux temperature.

18. The process of claim 15, wherein the solution is cooled to about room temperature.

19. The process of claim 15, wherein the obtained Rosiglitazone HBr Form I has a crystalline purity of at least about 80% by weight.

20. The process of claim 15, wherein the obtained Rosiglitazone HBr Form I has a crystalline purity of at least about 95% by weight.

21. Rosiglitazone HBr Form III, characterized by data selected from the group consisting of: a powder XRD pattern having peaks at about 4.5, 10.9, 17.0, 18.3, and 19.1±0.2 degrees two-theta; a powder XRD pattern with three peaks at about 10.9, 18.3 and 19.1±0.2 degrees two-theta and two peaks selected from the list of five peaks at about 4.5, 14.4, 16.3, 17.0 and 17.9±0.2 degrees two-theta; a powder XRD pattern with peaks at about 4.5, 10.9 and 19.1±0.2 degrees two-theta and additional peaks at about 16.3, 14.4, 17.0 and 17.9±0.2 degrees two-theta; a powder XRD pattern as depicted in FIG. 1; and combinations thereof.

22. The Rosiglitazone HBr of claim 21, characterized by a powder XRD pattern having peaks at about 4.5, 10.9, 17.0, 18.3, and 19.1±0.2 degrees two-theta.

23. The Rosiglitazone HBr of claim 21, having an X-ray diffraction diagram substantially as depicted in FIG. 1.

24. The Rosiglitazone HBr of claim 21, having a crystalline purity of at least about 80% by weight.

25. The Rosiglitazone HBr of claim 24, having a crystalline purity of at least about 90% by weight.

26. A process for preparing Rosiglitazone HBr Form III of claim 21 comprising providing a mixture of Rosiglitazone and water; heating the mixture to about reflux; admixing the mixture with a source of hydrobromide to provide a solution; and cooling the solution to obtain a precipitate.

27. The process of claim 26, wherein the obtained Rosiglitazone HBr Form III has a crystalline purity of at least about 80% by weight.

28. The process of claim 26, wherein the solution is cooled to a temperature of about 0° C. to about 60° C.

29. Method of treating diabetes comprising administering a therapeutically effective amount of Rosiglitazone hydrobromide form III, or IV as in claims 1 or 21, or Rosiglitzone hydrobromide form I as obtained in claim 15 to a patient in need thereof.

30. A pharmaceutical composition comprising Rosiglitazone hydrobromide form III or IV as in claims 1 or 21, or Rosiglitazone hydrobromide form I as obtained in claim 15.

31. A composition comprising at least 80% by weight of Rosiglitazone hydrobromide Form III as defined in claim 21.

32. The composition according to claim 31 wherein the composition contains 20% or less by weight of any other crystalline or amorphous form of Rosiglitazone hydrobromide.

33. The composition according to claim 32 wherein the any other crystalline form of Rosiglitazone hydrobromide is Form I as obtained in claim 15 or Rosiglitazone hydrobromide Form IV as in claim 1.

34. A composition comprising at least 80% by weight of Rosiglitazone hydrobromide Form IV as defined in claim 1.

35. The composition according to claim 34 wherein the composition contains 20% or less by weight of any other crystalline or amorphous form of Rosiglitazone hydrobromide.

36. The composition according to claim 35 wherein the any other crystalline form of Rosiglitazone hydrobromide is Form I as obtained in claim 15 or Rosiglitazone hydrobromide Form III as in claim 21.
